(19) United States
(12) Patent Application Publication
(10) Pub. No.: US 2018/0230031 A1
(43) Pub. Date: Aug. 16, 2018

(54) BIOREACTOR FOR TREATING WATER FLUID(S) BY BIOMASS

(71) Applicant: WINFLOW WATER OY, Kotka (FI)

(72) Inventors: OLLI-PEKKA HAILA, Kotka (FI); DEWI HAILA, Kotka (FI)

(73) Assignee: WINFLOW WATER OY, Kotka (FI)

(21) Appl. No.: 15/513,648

(22) PCT Filed: Sep. 28, 2015
(86) PCT No.: PCT/FI2015/050643

§ 371 Date: Mar. 23, 2017

(30) Foreign Application Priority Data
Sep. 30, 2014 (FI) 20145850

(51) Int. Cl. C02F 3/12; C02F 3/28; C12M 1/00; C12M 1/02

(52) U.S. Cl. CPC C02F 3/121; C02F 3/1236; C02F 3/286; C02F 3/2866; C02F 3/2846; C02F 2301/046; C12M 27/00; C02F 2203/004; C02F 2203/006; C02F 2209/001; C02F 2209/40; C12M 23/58

(57) ABSTRACT

The invention relates to a bioreactor for treating water fluid(s), and/or for producing a desired end product by biomass and/or for producing biomass. The invention relates also to methods for manufacturing and using such a bioreactor. The bioreactor (BR) includes at least a first processing unit (Z_{F}), a second processing unit (Z₂), a last processing unit (Z_{L}), and, optionally, additional processing unit(s) (Z₃, Z₄) between the second processing unit (Z₂) and the last processing unit (Z_{L}) in a plug flow configuration; at least one forward circulation system (FCS, FCS1, FCS2) for circulating biomass (BM) from the first processing unit (Z_{F}), to the last processing unit (Z_{L}) and/or to additional processing unit(s) (Z₃, Z₄); and at least one reverse circulation system (RCS, RCS1, RCS2) for circulating biomass (BM) from the last processing unit (Z_{L}) and/or from the additional processing unit(s) (Z₃, Z₄) to the first processing unit (Z_{F}).

### CROSS-REFERENCE TO RELATED APPLICATIONS

This application is a § 371 filing of PCT application PCT/FI2015/050643 filed on Sep. 28, 2015, which claims priority from Finnish application FI 20145850 filed on Sep. 30, 2014. The disclosures of these applications are included by reference herein in their entirety.

### BACKGROUND OF THE INVENTION

### Field of the Invention

This invention relates a bioreactor for treating water fluid(s), and/or for producing a desired end product by biomass and/or for producing biomass. The invention relates also to method for manufacturing and using such a bioreactor.

### Description of Related Art

Microbes are assumed to control the intake and excretion of nutrients and other chemical compounds by the changes in their enzyme levels. The levels are altered in accordance to the DNA through complicated mechanisms. For example, in the abundance of multiple nutrients simultaneously, the microbes select the most energy efficient or otherwise most preferable nutrient and exploit that to almost zero level before focusing processing on the next preferable nutrient. Studies have shown the enzyme levels during such selection to alter in such way that building the enzyme takes some time (this forming also a part of lag time), but the decay of such nutrient selecting enzymes is much slower, even after the nutrient is fully exploited.

The biofilm forms a majority of all biomass in a bioreactor (about 90% or so). The microbes and their growth are mostly concentrated at the layer closest to the surface of the biofilm. Thus, deeper inside a biofilm, floc or layer (100 μm and beyond), the biofilm forms a kinetics limitation to availability of nutrients and also disposal of excreted material, which are relatively slowly diffused through the biofilm layer to the surrounding fluid.

The most active portion of the biomass, and highest microbe density, is known to be very close to the surface of the biofilm or floc. It is assumed that the optimum depth can be as low as below 30-50 μm.

Many organic compounds are broken down in steps, and often this is performed by different microbe populations participating in different phases of the process. While the formation of larger flocs may provide the benefit of wider spectra of populations, the kinetics is also strongly reduced, which may become a more limiting factor than the benefit achieved for large floc sizes or layer thicknesses.

The growth rate, which can be seen as nutrient consumption rate multiplied by biomass yield, is known to increase with higher substrate concentration.

In the document US 2014238932 A1 there is disclosed a bioreactor system for the biological purification of wastewater comprising a tank, a dissolved air flotation system, a biological treatment unit and a clarifier in a plug flow configuration. In the material flow biomass is recirculated in a certain manner. In the document U.S. Pat. No. 5,514,278 A there is disclosed a bioreactor for process fluid and biomass treatment wherein process fluid is arranged to flow downstream and biomass upstream. The bioreactor comprises multiple processing units through which the material passes. The units receive biomass from the next adjacent units, while the process fluid is moved into the succeeding stage. The document suggests transferring biomass downstream bypassing one or several process stages, but does not disclose a forward circulation system for circulating biomass directly from the first unit to last unit.

### DESCRIPTION OF THE INVENTION

What has now been invented is a bioreactor for treating for treating water fluid(s), for producing a desired end product by biomass and/or for producing biomass with increased processing speed. The invention relates also to a method for the manufacture and use of such an apparatus.

The bioreactor of the invention and method for the manufacture and use of such a bioreactor are presented in independent claims. In addition, a few preferred embodiments of the invention are presented in dependent claims. The features recited in dependent claims are mutually freely combinable unless otherwise explicitly stated.

A bioreactor BR for treating water fluid(s) WF, and/or for producing a desired end product by biomass and/or for producing biomass, comprises at least first processing unit Z_{F}, second processing unit Z₂, last processing unit Z_{L}, and optionally additional processing units Z₃, Z₄ between second processing unit Z₂ and last processing unit Z_{L} in a plug flow configuration, at least one forward circulation system FCS, FCS1, FCS2 for circulating biomass BM from first processing unit Z_{F}, to last processing unit Z_{L} and at least one reverse circulation system RCS for circulating biomass BM from last processing unit Z_{L} to the second processing unit Z2 and from the second processing unit Z2 to first processing unit Z_{F}. According to an object of the invention, said bioreactor BR comprises at least four processing units. This adds adjustability and control of said bioreactor and possibility to use multiple FCS and/or RCS.

According to an object of the invention, said bioreactor BR comprises at least one biomass processing clarifier unit PCU. This allows use of even higher amount of biomass in the system while simplifying the secondary clarifier system.

According to an object of the invention, said bioreactor BR comprises at least one biomass modifying unit BMU. This adds possibility to modify the system biomass or temporarily use it outside of the system.

According to an object of the invention, said bioreactor further comprises at least one additional processing step added between first processing unit ZF and last processing unit ZL which at least partly participates to the processing of water fluid(s) WF but does not participate to main biomass circulation and/or forward circulation system FCS and/or reverse circulation system RCS. This adds possibility to modify the system for increasing processing speed and efficiency.

According to an object of the invention, the system is used to treat biomass present in the influent water fluid(s) or brought to the system otherwise

According to an object of the invention, at least one processing unit Z_{F}, Z₃, Z₃, Z₄, Z_{L} comprises at least one internal biomass clarifying unit ICU. This simplifies the system design and reduces pumping needs and can simplify the conversion of an existing system into an embodiment of the invention.

According to an object of the invention, internal clarifying unit(s) ICU comprises controlling channels CHA for self-adjusting water level and water fluid(s) WF and reverse circulating system RCS of biomass BM. This adds balance and stability to the system while reducing complexity.

According to an object of the invention, said bioreactor BR comprises at least two forward circulation systems FCS, FCS1, FCS2 and/or at least two reverse circulation systems RCS, RCS1, RCS2. This adds possibility to modify the system for increasing processing speed and efficiency.

According to an object of the invention, at least two processing units Z_{F}, Z₂, Z₃, Z₄, Z_{L} have been arranged at least partly to same vessel VES. This may reduce construction cost of the system.

According to an object of the invention, at least two processing units Z_{F}, Z₂, Z₃, Z₄, Z_{L} have been arranged into at least one plug flow vessel, where the at least part of the RCS is arranged to operate through diffusion and/or mixing of said vessel(s).

According to an object of the invention, at least one of the processing units Z_{F}, Z₂, Z₃, Z₄, Z_{L} have been arranged to form environmental conditions substantially different from other processing units, including but not limited to temperature, availability of substrate or dissolved oxygen and/or addition of chemicals, catalysts or enzymes. This adds flexibility and performance to the system when one or more processing steps are preferred to be performed in different environmental conditions than others.

According to an object of the invention, at least one of the processing units Z_{F}, Z₂, Z₃, Z₄, Z_{L} have been arranged so that at least a portion of the biomass thereof is either circulated outside of the system FCS and/or RCS or otherwise permanently removed for other function or purpose, including but not limited to nitrification, denitrification or production of biomass.

Said water fluid(s) can e.g. be or comprise fresh waters, process waters, waste waters, biomass and/or gas. Said bioreactor can be used as a bioreactor for producing a desired end product by biomass and/or producing biomass. Said bioreactor can be used for producing targeted end products, including but not limited to production of methane, ethanol or microbial biomass, or as a bioreactor for performing microbially a chemical reaction between at least two chemical compounds provided to the system, or as a bioreactor treating biomass present at the water fluid(s).

According to an object of the invention, the water fluid(s) is or comprise gas, while water is brought to the system along with the gas or separately. This allows processing of said gas, including but not limited to biological hydrogen sulfide (H₂S) removal from biogas.

A bioreactor according to the invention for treating water fluid(s) can now be utilized by smaller total HRT (processing volume), higher capacity for the same processing volume or better quality effluent, or a balanced combination of these. Different substrates of the influent water can be processed at least partially in different parts of the system, while the system tends to balance the processing along the whole length of a plug flow system. The system favors the most efficient microbes able to break down the influent substrates and also all generated intermediate processing products. Within the limits of the SRT, every kind of microbe required to process any given substrate in the influent is favored to ensure as complete processing as possible.

The system allows the use of higher MLSS, because biomass in the system is at least partially circulated internally, and only a fraction is flowing to the next processing step, such as filter, clarifier or other secondary or tertiary treatment. A higher MLSS allows further reduction of processing unit size, or improved processing performance. The system adjusts itself to having excess processing capacity reserve at its normal operating point, which allows higher peak loadings compared to conventional system.

The system also allows the processing to be performed more uniformly along the processing units, thus enabling more uniform aeration in aerated systems, more uniform processing profile for optimum total system volume and reducing phasing.

The faster growing microbes are dominating the surface of the biofilm (due to kinetics limitations), the further growing floc size may offset the benefit of spectra of populations. Furthermore, the biomass kinetics is in practice dominated by the effective surface area of such biofilm, and the surface area of a given total mass of flocs roughly doubles when floc diameter is half. Due to the above, it is beneficial to limit the floc size or biofilm thickness.

The biomass average residence time may define which microbes can have a significant population in the bioreactor, when the doubling time of a population exceeds the residence time of the biofilm, such population is far less likely to form a significant population count in such bioreactor. However, as referring to the biofilm floc size chapter, an improved kinetics of a biofilm due to smaller floc size or thinner layer depth may reduce the population doubling time. Thus, the residence time can be reduced when the kinetics is improved. The residence time of the biomass also affects the portion of viable biomass of the total biomass. Typically, the biomass can be divided to viable (active), dead, and lysed cells. These, together with all the non-biomass solids in the system form the total mass of solids in the system. As some portion of cells in a high biomass residence time system are bound to die instead of division (growth), which can be a result of kinetics due to large floc size etc., a shorter residence time of biomass typically improves the portion of viable cells compared to dead and lysed cells. The microbes can be seen as only consuming nutrients for growth. While the high biomass residence time endogenous system is perceived as using nutrients and energy only for cell repair, the same observations can be also viewed as growth through substrate consumption occurring at the same rate as the average cell death rate.

It has been observed that the microbes, while having high enzyme levels for certain nutrient, tend to collect an abundance of those nutrients inside their cell material while the environmental conditions for consuming the said nutrient are favorable. It has also been noticed, that for example when aerobic microbes are moved from good environment and higher nutrient levels to an anoxic state, the nutrients seem to be excreted after a relatively short lag time from inside the microbe cell to the biofilm and thus forward to the surrounding fluid.

The important outcome of such findings is, that when sufficient amount of microbes are brought from lower nutrient levels to higher nutrient levels, they absorb very fast the nutrients, until the nutrient level of the environment attains the same level as it was in the lower nutrient level. The nutrients are not fully processed immediately, and thus, the rate of nutrient absorption into the cell reduces over time and attains the consumption rate. Also, if the aeration is stopped momentarily, the nutrient level in the fluid after a relatively short lag time starts to rise.

Thus, it has been concluded, that typically the maximum nutrient or substrate consumption or absorption rate, to achieve for a given amount of biofilm/biomass in the system can be achieved, when
The enzyme levels for absorbing and consuming a substrate are already high in the microbes while the nutrients become available.
The biofilm is thin enough or has small enough floc size, to allow for maximum kinetics of nutrients, oxygen etc. and excreting of end products
The environmental conditions are otherwise favorable
New microbes which not yet have absorbed a saturation level of substrate inside their cells but have high enzyme levels for such substrate, are brought to the system position where high substrate reduction is desired
Microbes having already absorbed the substrate to the full inside their cells, are removed from the system or displaced within the system thus that they remain in otherwise favorable environmental conditions.

With this method, the said system can be made to remove significantly more nutrients from the process water than what the microbial consumption for maximum growth would normally allow.

### BRIEF DESCRIPTION OF THE DRAWINGS

Some features and advantages of the invention will be described in the drawings:

FIG. 1. Basic biomass circulation system with one loop.

FIG. 2. Overlapping circulation loops.

FIG. 3. Basic processing clarifier system, biomass or TSS leakage along with the process fluid.

FIG. 4. Processing clarifier system.

FIG. 5. Basic processing in plug arrangement.

FIG. 6. Basic biomass circulation system FCS-RCS for water fluid(s) WF with biomass modification unit BMU connected to first processing unit ZF.

FIG. 7. Basic processing in cross circulation.

FIG. 8. Principle for simple countercurrent biomass movement in an aerated activated sludge reactor BR-AS configured as an embodiment of the invention.

FIG. 9. Practical detailed example of simple countercurrent biomass movement in an aerated activated sludge reactor BR-AS configured as an embodiment using ICU.

FIG. 10. A simplified example of a substrate pumping system.

### SYSTEM OPERATION

Basic System

The basic system configuration of the invention consists of a plurality, 2 or more, of processing units configured in a plug flow configuration, where the process fluid flow and the flow of active biomass are (mainly) counter current.

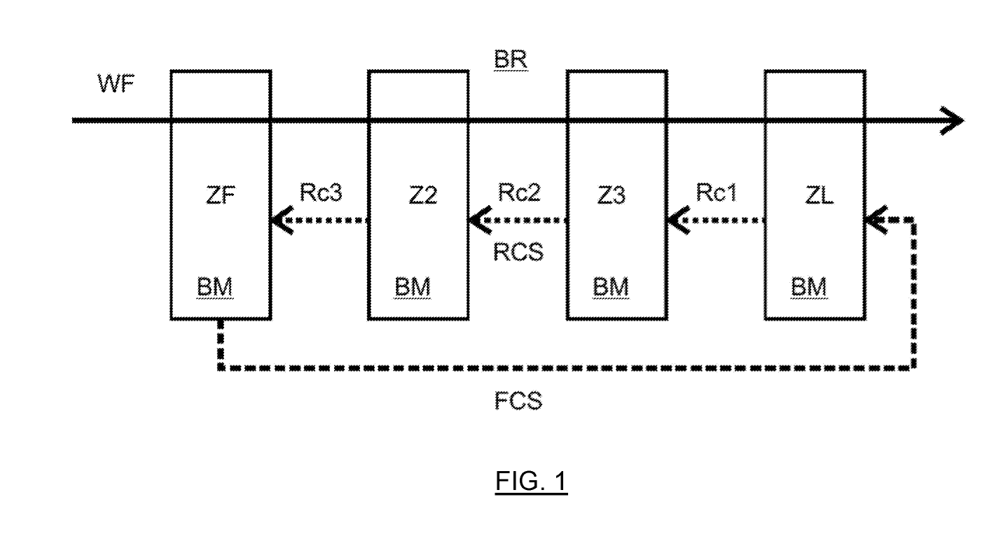

Basic system configuration, described in FIG. 1, can be used alone, or as a part of a larger system, number of actual units included in the loop may vary. In this system, the process fluid, water fluid(s) WF flows from first processing unit ZF to last processing unit ZL, while the biomass BM flows eventually from ZL to ZF and is returned from ZF to ZL. It is important to note that the net flow of biomass between Z2-ZF, Z3-Z2 and ZL-Z3 is against the process fluid flow WF, and it is moved from the first ZF to the last processing unit ZL as a return flow for example by pumping, moving biomass carriers, etc.

It is well known to those skilled in the art that the leakage of biomass and other solids along with the process fluid may partially compensate the biomass return from a later part of the system to an earlier part thereof. In new invention the net flow of biomass in the system is arranged against the normal direction of the flow of the process fluid between adjacent units.

Preferably forward circulation system is provided to move biomass from a first processing unit ZF at an average rate exceeding the growth of biomass in said first processing unit ZF subtracted by the rate of disposal of biomass from said first processing unit. Preferably biomass flow rate of reverse circulating system RCS or any part thereof between any two processing units included in the biomass circulation loop is provided to exceed the leakage of biomass along with the normal process water fluid(s) flow between said two processing units.

Implementation of the Biomass Flow

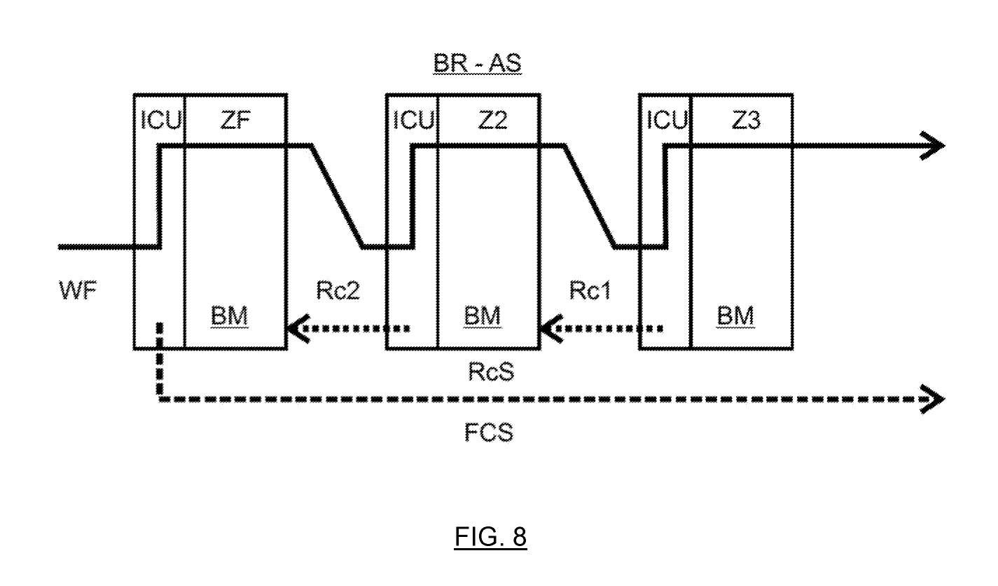

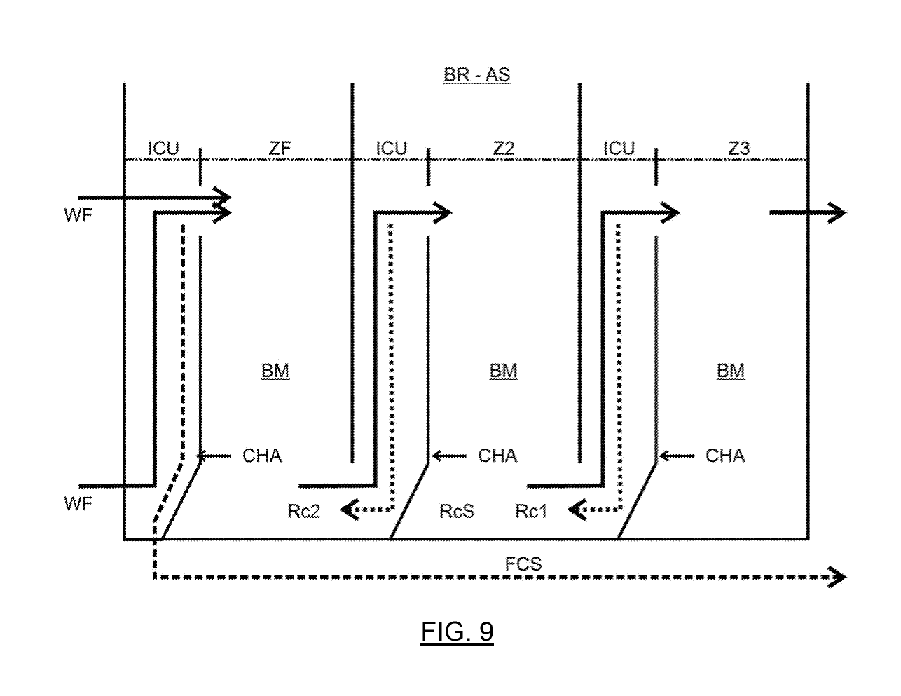

The circulation of biomass against the process fluid flow can be implemented including, but not limited to, by pumping or other means of actively transferring the biomass from a first unit to a second unit receiving the biomass using a means of separating it from the process fluid of the said first unit such as filtering or settling, by collecting biomass carriers or other means of biomass attachment or carrying vehicles from said first unit to said second unit, by using a configuration of FIGS. 8 and 9 or other similar configuration where the flow of biomass is arranged to be counter current to the process fluid flow by means of gravity, or as a combination of the aforesaid.

The biomass feedforward from the first unit participating to the biomass circulation in a biomass circulation loop to the last unit of said loop can also be arranged using similar or other method as the circulation against the process flow, while it may be beneficial to use a higher degree of separation of the biomass from the process fluid to limit short circuiting of the process fluid along with the biomass from said first unit to said second, receiving, unit, especially when the biomass circulation rate selected is relatively large compared to the process fluid flow rate or the biomass portion of such transferred combination of biomass and process fluid is otherwise relatively low, to reduce process fluid short circuiting.

However, it has also been verified that a system of high performance and low cost can be successfully implemented using for example processing units similar to FIG. 9 and a simple pumping of process fluid in the first unit participating to the sludge circulation loop to the last unit of said loop without implementing any means of separation.

Consumption of Influent Substrates in the Basic System

While in ZF, the biomass absorbs and partially consumes the substrate. When moved to ZL, it continues to process the substrate without releasing it, until it is depleted from inside the cell and biofilm, and its starts to adopt other available substrates to consume by altering its state, including building enzymes, for adoption to the most preferred available substrate.

In a normally working system, all influent substrate levels are at their minimum at ZL. If any type of substrate is available at higher quantities, the microbes try to adopt and absorb those to avoid starvation.

When moved to Z3, some substrates are available at higher concentrations than in ZL. Some enzymes built to very high or maximum level for absorbing and consuming certain substrates while in ZL, are now at relatively high levels, which allows the microbes to consume those at maximum rate with minimum or no a lag time penalty for adaptation.

Most substrates are still in relatively low level in Z3, as the incoming microbes from ZL are absorbing them at a rate higher than their consumption inside the cell.

When moved to Z2, similar phenomena occurs as in Z3, but typically a more abundant levels and spectra of substrates are available. However, in Z2 and Z3, the concentrations of most preferred influent substrates are typically low or almost depleted.

When moved to ZF again, the microbes select the most preferred substrate available in the influent, which likely exists in highest concentration in ZF compared to rest of the system.

It is also beneficial to the system that while in ZF and at abundance of substrate, the microbes typically use the substrate in an inefficient way, such as excess heat production and energy-spilling pathways. However, when arriving to ZL and being under conditions of severe constrains to growth, the microbes still continue to maintain high energy flux, as cell membrane energization and function of transport systems are essential conditions of resumption of growth whenever the environmental conditions change.

Thus the circulation of biomass as described also increases the microbial processing speed of the substrate compared to endogenous system.

Favoring of Microbial Populations

As the microbial growth rates depend on availability of substrates, the system in general favors those microbes which can consume the substrate at the fastest rate at the excess of nutrients, and at that perceived concentration of substrate produces the fastest rate of growth.

The microbes, which absorb into their cell the most nutrients at the shortest time (even if at excess), will perceive highest substrate levels, and thus the system also favors those.

Also, any nutrient which does not become the most preferred substrate at any place of the system for any other faster growing population, will provide an opportunity for the most suitable microbe preferring such substrate.

Thus, the system actively favors the microbial species spectra, which represent the fastest growth and substrate consumption rates for any given substrate available.

As all microbe populations are nearly uniformly represented in all processing units, the aforesaid is also applies to the intermediate products.

Furthermore, it may be desired that the environmental conditions in the processing units substantially change in one or more ways, such that different microbe populations may experience optimum conditions in different processing units, for example when said conditions in at least one of the processing units are anaerobic, anoxic or anaerobic, unlike in others. In such system the processing can be phased or otherwise controlled and the consumption of substrates can be altered in accordance with the requirements set to the system.

Intermediate Products

Some of the substrate in the influent is typically biodegraded in more than one step, or performed by more than one microbe population. Such biodegradation process produces one or more intermediate products, whose concentration varies in the system, depending on multiple parameters.

In general, the intermediate products can be seen as new substrates introduced to the system at the place where the processing or consumption of the original substrate takes place.

Without the biomass circulation, this phenomena may lead to phasing of the whole system, and thus may give rise to insufficient processing times for such intermediate product, as well as inhibition of processing in some parts of the system.

This basic system also distributes the processing of each substrate, in order that the intermediate products are available more evenly uniformly along the system.

As a result, said intermediate products are absorbed and consumed properly. For example, intermediate products produced in ZL by microbes who have absorbed the original substrate at ZF and are moved to ZL, are likely yet at a far lower level than at Z3 or Z2, which means that the already high enzyme levels and absorbance capability of suitable microbes at ZL, which may be already near starvation, are effectively able to further process the said intermediate products.

Furthermore, such intermediate products are typically in greatest abundance in ZF, assuming that the original substrate of said intermediate product is preferred by microbes in ZF.

As to any intermediate product, the earlier along the process fluid flow where such original substrate is converted to such intermediate product, the longer processing time is available for such intermediate product to be reduced further. Furthermore, as the microbes absorbing and consuming such product are continuously moved towards the front of the system, it can be said that the longer the processing of such intermediate product takes, the longer processing time the system allows for such substrate, and thus, all the process steps of biodegradation of any (biodegradable) substance are given more processing time than in a plug flow system without biomass circulation,

The basic system can be implemented as aerobic or anaerobic processing system. However, when at least part of the aerobic system has anoxic phases, especially the strictly aerobic microbes may excrete some of the nutrients to the surrounding biofilm and fluid, resulting in increased substrate levels in the process fluid.

The same may be observed in anaerobic microbes exposed to aerobic conditions.

While the negative effects of this phenomena can also be reduced in the basic system, it may yet be preferable in the system implementation to ensure that a flow between aerobic/anoxic/anaerobic parts of the system is implemented such that only the process fluid is moved between states, and the biomass exchange between said parts is reduced.

Multiple Circulation Loops

In some cases, more than 1 circulation loop can be used to provide additional benefits in the system. Such benefits may include for example alteration of aerobic and anaerobic processing resulting in order to lower the aeration energy required, enable proper nitrification/denitrification performance etc.

The multiple overlapped loops also allows intentional (partial) phasing of the system, when that is desired, while retaining many other benefits of the system.

The benefit of multiple biomass circulation loops in this example application comes mainly from the vastly improved processing time in both aerobic and anaerobic processing, which allows shorter system HRT and thus smaller total physical volume for the system without increasing the process temperature. However, the immunity to phasing especially in the anaerobic processing side is another key benefit in terms of process stability and controllability.

Multiple Overlapping Loops

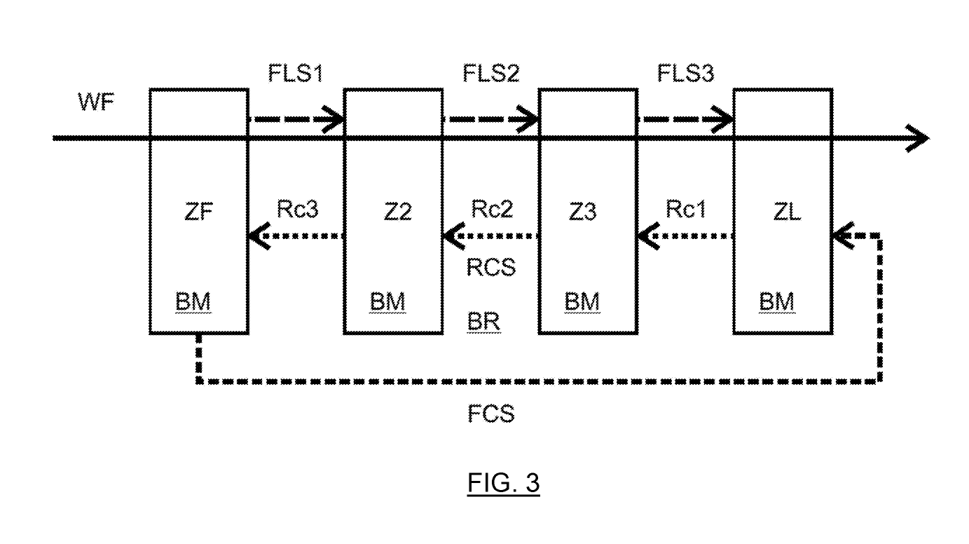

Multiple overlapping loops can be configured in a system. Such system can be configured to suit special needs, for example where the influent has significant amounts of slowly biodegradable substrate. Overlapping circulation loops are described in FIG. 3.

The example in FIG. 3 can have unique benefits, as it allows full recirculation of biomass BM while ensuring that the biosolids attached to the biomass in ZF will have a minimum flow through all the system parts prior to becoming part of the effluent; i.e. the minimum path is ZF→Z3→Z2→ZL, and the forward and reverse circulation rates of loops 1 and 2 can influence the average residence time in the system for such biosolids, provided that the biomass flow along with the process fluid flow is sufficiently controlled.

Moving the Biomass in the Circulation

In the given examples, it is assumed that the flow of biomass counter current to the process flow is arranged between the process vessels. This also ensures biomass retention and balance in each vessel to a sufficient amount.

The biomass flow from the Z_{F} to Z_{L} or between other parts of the process can be arranged by simple pumping.

If two or more of the processing units are arranged for example as one or more long plug-flow vessel, where the number of individual processing units can be seen high while a physical boundary between said units arranged as one vessel is reduced even to diffusion and gravity flow, the FCS and/or RCS removing biomass from an earlier part of said vessel and/or to a later part of said vessel creates a biomass concentration gradient within the said vessel. The gradient will reduce through mixing and/or diffusion, thus forming the reverse circulation of biomass from the later part of said vessel to an earlier part and acting as at least part of an RCS within the said vessel and/or forming at least one RCS between the processing units within the said vessel.

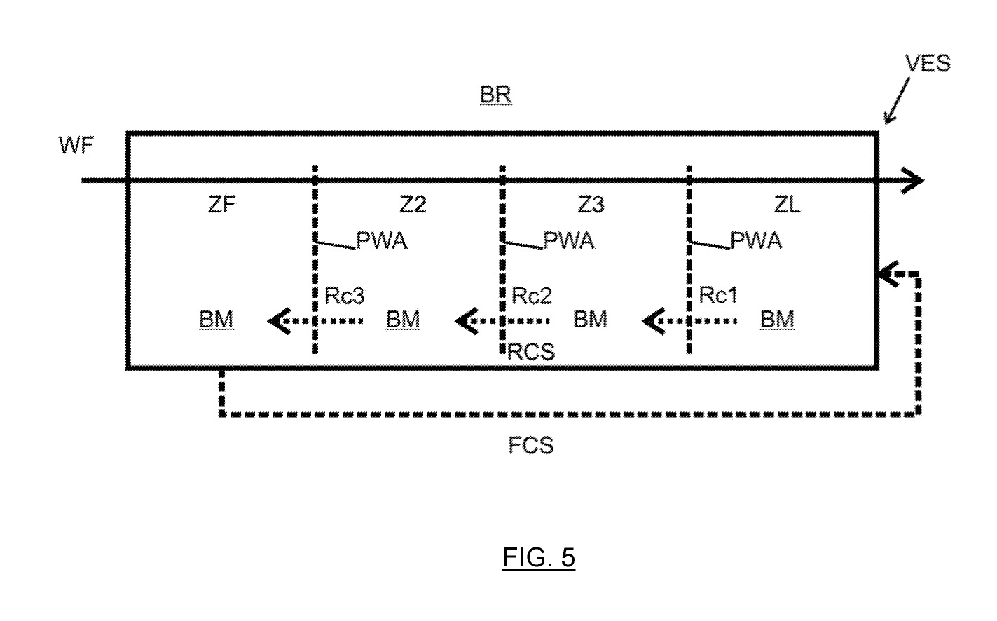

An example of such configuration is given in FIG. 5. In this example, the whole system is arranged in one vessel VES, with partial walls PWA separating processing units ZF, Z2, Z3 and ZL. The partial wall allows the flow of WF through the PWAs from ZF to ZL, and also allows the biomass BM flow through the PWAs from ZL to ZF. The FCS moves the biomass from ZF to ZL at a rate higher than the growth of biomass in ZF, causing a concentration gradient of biomass, which induces a net flow of biomass from ZL towards ZF via Z3 and Z2.

The partial wall PWA forms a resistance to biomass flow through reducing free mixing and diffusion between the units. This resistance is a function of the area of opening of such wall and the flow of WF through the said opening, as the two are counter current. A similar resistance to biomass flow at a given flow of WF can be achieved when the opening is formed as a channel with known surface area and length.

In this kind of configuration, when no other additional form of RCS is used between the processing units, the biomass concentration is highest at the ZL, and lowest at ZF, which will result in inefficient use of total system volume or inferior system performance compared to for example by constructing a simple ICU in the unit(s).

If a normal AS system is being modified into an embodiment of the invention, the reverse circulation of biomass can be preferably achieved cost efficiently for example in the configuration of FIGS. 8 and 9.

In FIGS. 8 and 9 an activated sludge reactor BR-AS comprising ICUs is shown. Any pair of two adjacent units, for example ZF and Z2, or Z2 and Z3, with said pair abbreviated here as Zx and Zy, where at least Z_{Y} comprises an ICU with channel CHA through which the communication occurs such that the process fluid WF can freely flow from Z_{X} to Z_{Y}, but simultaneously the CHA acts as a simple clarifier for Z_{X}, where the biomass (BM) entering CHA is returned to Z_{X}. When any number of units Z_{F}. . . Z_{L} is arranged in a plug flow configuration in a similar way, the net biomass flow is towards the first unit ZF, i.e. counter current to the process fluid flow. The channel CHA forming the port between the aerated portions of any two adjacent units Z_{X} and Z_{Y} resists the biomass BM flow with the process fluid WF, while being open at the top part of the Z_{Y}, the top of the channel CHA receives biomass BM from Z_{Y} and effectively transports it to the previous unit Z_{X} of the process flow WF.

This channel with its ports is an example embodiment of the internal clarifier unit ICU of Z_{Y}.

This configuration is often sufficient for the purpose, even if some amount of turbulence is present in the channel, but where it poses a problem, improving the settling of biomass or reducing the residence time in the channel can be easily arranged for example in ways similar to those used in settlers.

The amount of biomass transferred from Z_{Y} to Z_{X} depends strongly on firstly the amount of process fluid of Z_{Y} (along with the biomass within) exposed to the upper part of the CHA and thus subjected to biomass separating/settling down and through the channel, and secondly, the concentration of biomass in the Z_{Y} process fluid. The former can be largely selected as a mechanical design parameter and also self-regulating the latter, the biomass concentration in Z_{Y}. Thus, the biomass concentration in each processing unit Z_{Y} can be set individually by design, as well as the average SRT of biomass in each processing unit Z_{Y}, independently of its process fluid volume or flow.

An implementation similar to FIG. 8 can be implemented when the biomass is settling down by gravity in the process fluid. If the biomass will normally float, as it would when for example attached to a floating biofilm carrier, a similar channel can be used reversed.

The parameters related to biomass leakage rate from Z_{X} to Z_{Y} and biomass return rate from Z_{Y} to Z_{X} may depend on for example settling properties of the biomass and can be influenced by design.

In some cases, the ICU may be preferred to be used in a modified way. For example, in Z_{F}, it may be beneficial to not bring the influent through the CHA, but only use the CHA to separate or condense biomass for the FCS. Thus the influent short circuiting to Z_{L} can be prevented or reduced.

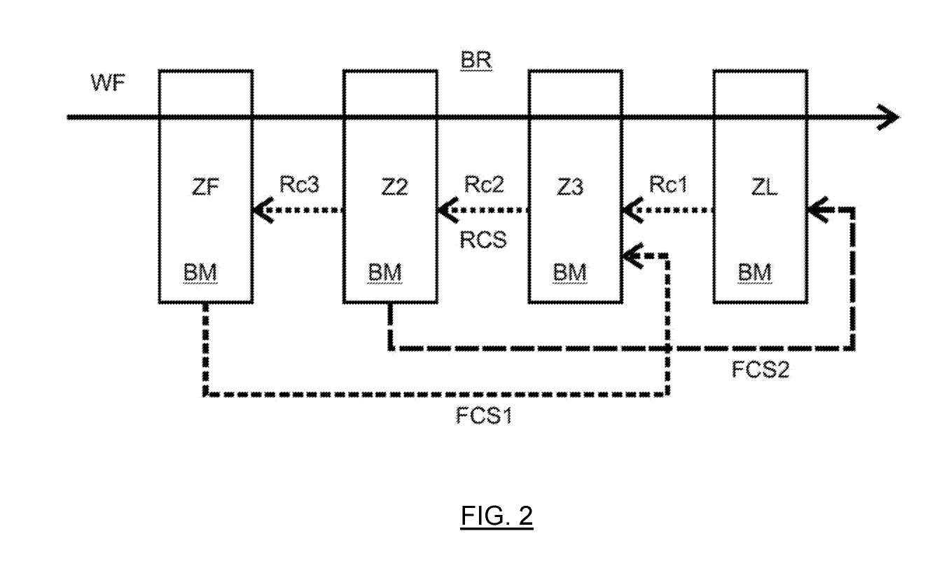

Also, in the case where the two adjacent units are desired to have different biomass, for example in a system of FIG. 2, a CHA in Z3 can be used without port to Z2, to separate or condense biomass for the purpose of implementing RCF2. Also in Z3, a separate CHA can be used with modification to prevent biomass flow from Z3 to top of said modified CHA and thus through said modified CHA to Z2, such that said modified CHA blocks biomass flow both ways. This method may be preferred to keep the biomass loops separated.

A similar type of configuration can be arranged for example between anaerobic or anoxic continuously mixed processing units. Also, similar type of configuration can be arranged when the said two processing units comprise a combination of aerated, non-aerated, still or mixed processing units, and also when the said combinations are at least partially intermittent in their operation, for example when the processing unit operates in batch modes, for example batch aeration and/or batch process fluid flow.

In a batch operated system, it may be beneficial to use the same configuration as reversed, such that the channel of flow to the next processing unit is at the top of the first unit and bottom of the next unit, such that at settling phase the biomass is concentrated at the bottom of the unit, and the flow at the fill phase, after the settling phase does not transfer biomass to the second unit. In such batch system, it may be beneficial to also reduce the amount of biomass entering the channel during mixing or aeration phase, as it would transfer biomass forward in the system between adjacent units, which is not desired.

In such batch system configuration also the FCS and RCS can be easily arranged by for example by selecting the channel width such that a biomass transfer counter current to the normal process fluid flow occurs by pumping at the same channel, but opposite direction to process fluid flow. With such configuration, for example instead of fill phase, biomass is pumped after settling from ZF to ZL, and the created gravity potential causes a flow in all units from ZL towards ZF, and the biomass thus flows through the channels towards ZL.

In a plug flow anaerobic system, such as UASB, retention of the biomass is already implemented in the normal system, and circulation could be arranged simply by pumping some of the biomass residing in the bottom of the reactor to the upper parts of the reactor, where the rest of the circulation towards the bottom part of the reactor is completed by gravity.

Biomass Circulation Rate

If we assume that the channel can retain for example 80% of the biomass from moving forward in the system, and similarly 80% of the biomass entering the top of the channel due turbulences flows through the channel, we can assume that the probability of biomass moving forward in the system (against desired direction) is only a few percent. Also, as the rate of biomass moving against the process fluid flow can be reasonably well controlled to approximate the desired value, a total average biomass recirculation time can be established and set to a desired value.

Selecting the optimum circulation rate depends on many parameters, including but not limited to such as system HRT and selected SRT, the nature of biodegradable material and its biodegradation speed and the efficiency of the biomass retention between the units, and also the excess sludge disposal rate and mechanism.

It is also possible through suitable design to implement for example an RCS where the return rate of the biomass varies between processing units over time or by biomass concentration. This allows the selection of different retention time for biomass in different processing units.

Also, for example in an aerated system, mixing or biomass circulation can be intermittent, or otherwise be arranged as a function of time or other parameter, including but not limited to reasons of OLR, HLR, nitrogen or phosphorous removal, etc.

Due to the aforesaid, the biomass circulation rate in some configurations is also selected by design based on parameters including but limited to, the system configuration, selected residence times in each processing unit and the positions and rates of one or more excess biomass removal mechanisms.

Excess Biomass Removal

In the system of the invention, the substrate removal capacity for a given total amount of biomass may be several times higher than a normal system. While biomass yield per amount of COD processed, especially in an aerated system, can be lower than conventional, the biomass SRT will typically reduce.

A very short HRT is often desired, and therefore to ensure that all the microbial populations required by the process can form a sufficient population using the selected SRT, may give further limitations or guidance to the system, such as selecting increased biomass quantities or higher MLSS values in an activated sludge process.

In for example an activated sludge system, the excess biomass removal can also be implemented to the biomass FCS, thus that the biomass being pumped from Z_{F} is divided into separate flows, where one flow enters the Z_{L}, and another flow is directed to an excess biomass handling system, such as sludge thickening.

Another means of implementing suitable biomass circulation and/or removal rate can utilize the configuration similar to that of FIGS. 8 and 9. It has been observed especially in an activated sludge system that in the channel of communication between two processing units of FIGS. 8 and 9 employs a property that the biomass concentration in the aerated or mixed processing unit before the channel influences the height of the biomass blanket formed in the channel. Thus, the amount of biomass in the processing unit can be controlled with the means of removal of the biomass at a selected height in the following upflow channel. Furthermore, when the biomass is removed at the top of said blanket, the removed biomass is selected based partly on its settling properties, thus the better settling biomass is retained in the process.

Accordingly, as the system biomass in a biomass circulation loop is circulated between all the units participating to such loop, the removal of excess biomass can be implemented virtually in any of the channels in a system with FIGS. 8 and 9 type of configuration.

If the excess biomass removal in a biomass circulation loop is implemented for example in the first processing unit ZF of said loop, the biomass residence time in latter processing units can also be made longer than in processing unit ZF. Thus, the environmental cycle of the biomass between high and low substrate conditions can be selected.

An even larger freedom of selecting different SRTs and environmental conditions individually for different processing units of a system can be achieved when more than 1 loop is used.

Biomass in Biomass Carrier System

The biomass FCS and RCS implementation in a moving biofilm carrier system is quite straightforward in terms of an application as the biofilm carriers are typically easy to separate from the process fluid. The rate of biomass movement can also be relatively accurately defined. However, the mechanism of biofilm carrier movement needs to be selected based on the carrier type selected.

Basic processing can have biomass and/or TSS leakage along with the process fluid, as shown in FIG. 3. The FLS1, FLS2 and FLS3 represent the leakages along with the processing fluid flow. These leakages are opposite to the desired biomass direction of movement, and partially reduce the net biomass flow. It is beneficial that the leakages are reduced or minimized, and also it is important that the Rc3, Rc2 and Rc1 as parts of the RCS, compensate the leakages FLS1, FLS2 and FLS3, respectively, such that the net flow of biomass remains against the process fluid flow.

Biomass Modification

Depending on the nature of the biomass in the circulation, for example when moving biomass carriers are used, it may be beneficial to clean the carriers or other media circulating within the loop. Also, for example when activated sludge is used, it may be beneficial to alter the composition of the sludge or select the circulated sludge based on its settling properties. It is also beneficial to allow the biomass excrete the nutrients absorbed inside the cell prior to returning to the main process flow, for example to reduce the main flow HRT. Furthermore, for example anaerobic or anoxic sludge can be temporarily used outside of the main biomass circulation for denitrification.

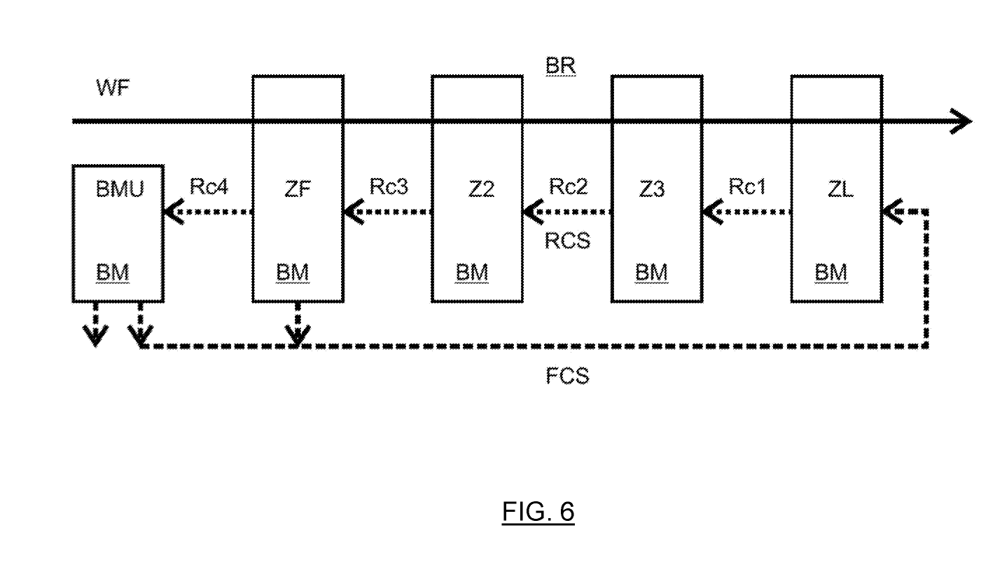

An example of a biomass modification positioned in the biomass return from the first to the last unit in the loop is given in FIG. 6.

The biomass modification unit BMU is a system or subsystem where the biomass is temporarily moved from the biomass circulation loop for some other purpose than normal processing of the process fluid normally occurring in the said loop.

The BMU can also be configured as a means of excess biomass removal.

It has been discovered during further study of the invention, that when for example aerobic processing is used and the biomass participating in the loop is temporarily moved from an aerobic processing to an anaerobic or anoxic vessel, together with the process fluid, the substrate concentration of the moved processing fluid containing said biomass rises fast after a short lag time.

This phenomena can be seen as the aerobic microbes excreting the substrate to the surrounding fluid when the DO or other environmental prerequisites for normal growth are suddenly limited. This phenomena is characteristic to the invention, but does not occur significantly in for example AS system clarifier, partially because the microbes in the AS system are in endogenous phase and thus they do not have excess substrate inside their cell.

However, the system of the invention encourages microbes in the later parts of the system, while in depletion of substrates, to modify their enzymes or other mechanisms for absorbing substrate inside their cell at maximum efficiency, and when moved to earlier, or especially to the first unit of the system, the microbes still are absorbing substrates at highly elevated rates.

It has been observed, including but limited to the means of oxygen uptake rate (OUR) compared to influent substrate flow into the first unit, that in the first unit of the system the microbes absorb significantly more substrate than they can consume.

It has also been observed, that this phenomena occurs at least significantly less in the later, especially the last unit of the system. Therefore, it is beneficial that for example after the last unit of an aerobic biomass loop, the loop biomass can be exposed to anoxic or anaerobic conditions without significant return of substrate back to the processing fluid.

The biomass modification unit BMU may be connected to any one or more units ZF, Z2, Z3, ZL in the biomass BM circulation loops FCS, RCS (Rc1, Rc2, Rc3, etc.).

A practical application for such BMU can also be removing some undesired influent contamination, substrate or intermediate products, for example separation of solids, fat, oil or grease. When the environmental conditions in the BMU are different or more harsh in terms of DO, shear, other variation of the normal condition or a combination of those, certain substances can be separated from the biomass, the biomass can be selected based on its properties, or biofilm carriers can be cleaned or their excess biofilm can be partially removed to reduce its thickness.

Another application of BMU is also using the microbes in the loop for substrate pumping; when for example the aerobic microbes are exposed to anoxic conditions in the biomass modification unit, it has been discovered that a significant portion of the influent substrate can be extracted from the biomass to the fluid in the biomass modification unit.

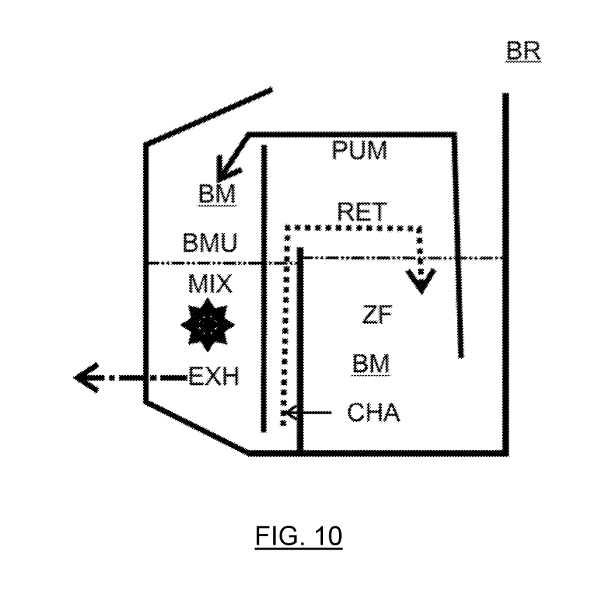

A simplified example of a substrate pump configuration is shown in FIG. 10. The BMU is configured as an anoxic biomass modification unit and at the start of a cycle receives a batch of mixed process water and biomass BM from ZF. After pumping is stopped, the mixer keeps the biomass in suspension for a selected time, such as 30-120 minutes, after which the settling starts. After settling, the settled biomass is returned to ZF, while the process water enriched with substrate from the biomass BM is moved to anaerobic processing.

The unit can be driven for example in the following scheme of phases:
1. The biomass BM from ZF is pumped PUM into the biomass modifying unit BMU. While pumping, the previous batch of biomass is flushed back RET to ZF through the overflow channel CHA.
2. The mixer MIX starts and keeps the biomass in anoxic or anaerobic suspension for example 30-120 minutes.
3. After mixing phase, the settling of biomass starts and after sufficient settling time, most of the good settling biomass is below the pumping level at preferable level.
4. The pumping starts and moves the process water enriched with the substrate excreted by the biomass EXH e.g. to anaerobic processing.

Returned biomass RET flushed back to ZF will continue its normal cycle there.

The system has 3 major benefits; firstly the substrate can be processed anaerobically, secondly the biomass returned to the main loop will have significantly lower new biomass yield than without such growth phase interrupting cycle, and thirdly also the biomass which has inferior settling properties can be extracted from the main loop and will be digested anaerobically, which also improves the settling properties of the main process flow when entering the secondary clarifier.

Thus, if the aerobic biomass in the anoxic biomass modification, after extraction of the substrate to the fluid thereof during the mixing phase, is moved back to the loop while the fluid remaining in the biomass modification unit BMU is moved to anaerobic processing, significant cost savings can be achieved. The anaerobic processing is more energy efficient and its flow is independent of the processing fluid flow. Also an elevated temperature for such anaerobic processing can be used efficiently for relatively low substrate concentration influents. Thus it is economically feasible to process such part of the influent substrate in an anaerobic system separate from the main process fluid flow.

A Processing Clarifier

A processing clarifier system can be aerobic, anoxic or anaerobic system where the biomass is moved towards ZL and/or other earlier parts of the system against the processing fluid, and where processing or post processing takes place in the units simultaneously with the clarifier function.

This is beneficial for the secondary clarifier operation, and in some cases the separate secondary clarifier is not needed. Due to the nature of a system with high biomass retention in each unit, the system can be configured to have relatively high MLSS, when after the last processing unit there is implemented one or more processing clarifier units, mainly for biomass retention and return.

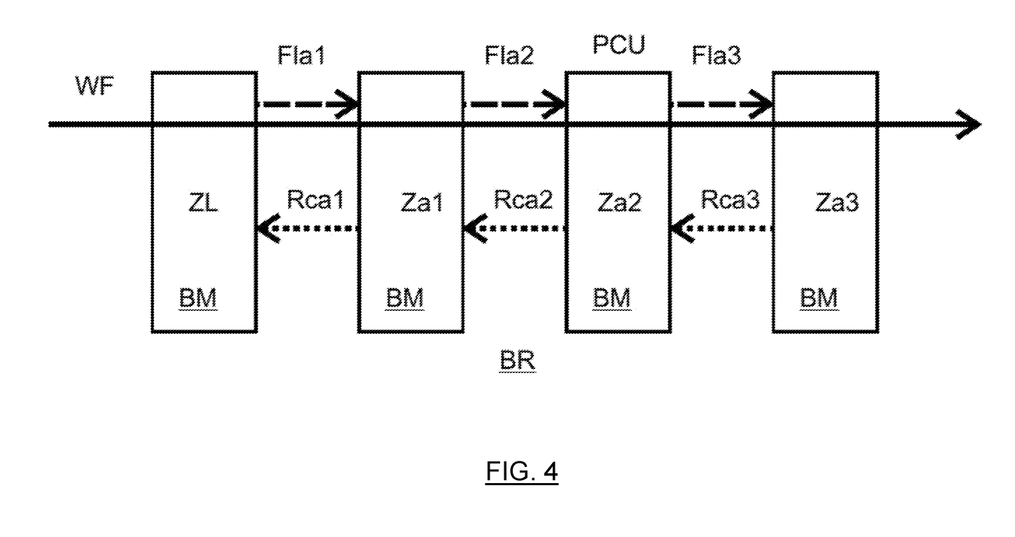

An example of a processing clarifier system is given in FIG. 4. The units Za1, Za2, Za3 are configured as processing clarifier units PCU after the last processing unit ZL of the biomass circulation system. In practice, the RCS type system is implemented without FCS and thus all biomass is moved towards ZL. The leakages of biomass Fla1, Fla2 and Fla3 are overcompensated by reverse circulation units Rca1, Rca2 and Rca3, respectively.

It is important to notice that for example if the return rate of Rca1 is selected such that the system reaches balance between biomass transfer rates of Rca1 and Fla1 when the biomass concentration in ZL is 3 times that of the biomass concentration in Za1, and all stages of Za1, Za2 and Za3 are similar, the total leakage of biomass from ZL to secondary clarifier can be reduced to below 4% of ZL concentration.

Such processing clarifier units could be seen as a pre-clarifiers prior to secondary clarifier, each of which retaining or returning against the flow majority of their biomass, while simultaneously acting as small processing units.

For example, such pre-clarifiers can be similar to the configuration of FIGS. 8 and 9 and thus have an ICU, with the actual “processing volume” reduced to small or minimum, and where HRT is in practice dominated by the channel HRT. Thus, the MLSS of the preceding system units, especially Z_{L}, can be selected practically without secondary clarifier limiting the MLSS selection.

Another specific benefit of said processing clarifier unit PCU for example in aerated systems is that the microbes with high amount of absorbed nutrients will not release said nutrients to the biofilm and thus to the effluent, as the environment remains favorable.

While the required SRT to ensure the population levels of all desired microbe populations may otherwise limit the achievable rate of biomass disposal, the units in such system may contain other media than that of the prior processing, such as fixed or moving biofilm carriers.

One application is to introduce an anoxic or anaerobic processing after an aerobic biomass loop, where the biomass leaking from the previous stage is returned to such previous stage. Beneficially, when for example fixed or moving media is used inside the processing clarifier units for anaerobic or anoxic processing, such as denitrification, also the biomass separated from such fixed or moving media will be returned to the prior loop and disposed of together with the loop biomass disposal.

Another application is to establish an aerobic post processing or polishing with for example fixed or moving biofilm carriers after anaerobic digestion stage, where the separated biomass is moved to the earlier stage of the system thus digesting also the aerobic sludge generated in the polishing stage. If for example moving biofilm carriers are used in the processing clarifier units, a biofilm circulation loop of the invention can be arranged for such carriers in the processing clarifier system to improve the processing performance.

Return Sludge

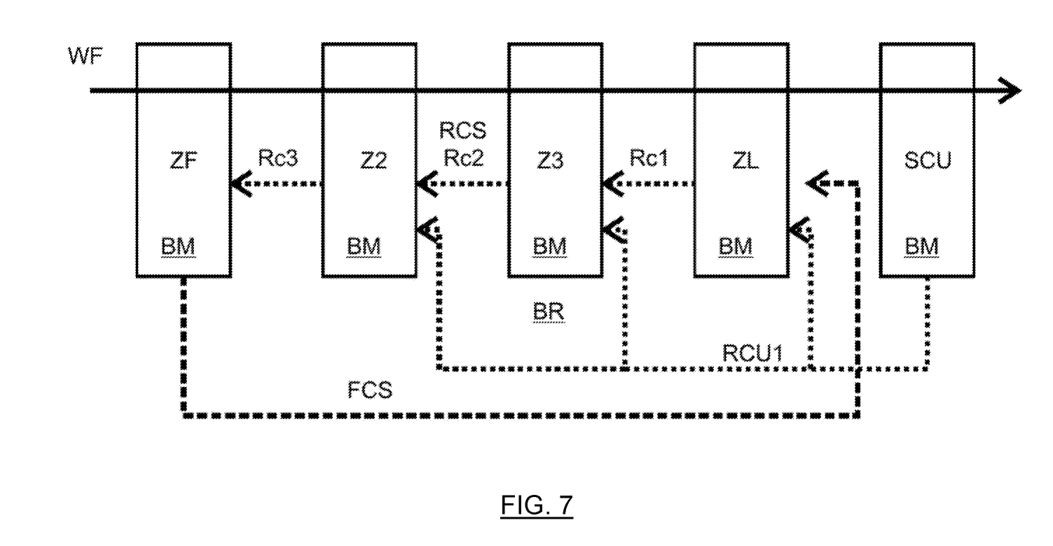

In FIG. 7 a system with secondary clarifier unit SCU is presented with return biomass circulation unit RCU1 returning the biomass from the SCU. Unlike in the conventional system, it is beneficial to not arrange the biomass return from SCU directly to ZF, because the microbes may have been exposed relatively long in the SCU to environmental conditions from which the microbes are not able to resume normal metabolism immediately. Therefore, it may be beneficial to return the biomass through the shown alternative routes to Z2, Z3 or ZL instead of ZF.

At least a portion of the biomass from the SCU can also be disposed of, especially if a processing clarifier unit PCU is used between ZL and SCU.

System Requirements for the FCS and RCS

The BM balance in all processing units must be maintained at the desired level. The balance between the processing units can be set in the design of the RCS. The total amount of BM in Z_{F} without FCS or RCS will change at the rate of BM net growth in Z_{F} (rGrowth), rate of BM entering along with the influent (rInfluent) and rate of BM leaking from Z_{F} forward along with the process fluid (rLeak) and rate of BM removed from the system at Z_{F} (rRemoved), such that the net rate of BM change in Z_{F} is

rNet=rGrowth+rInfluent−rLeak−rRemoved

Also, the RCS is moving BM to the Z_{F} at the rate of rRCS. The rRCS is one of the key system design parameters, as it defines the amount of BM coming from lower nutrient environment entering in Z_{F} and thus it significantly influences the nutrient absorption rate at Z_{F}.

It is important to notice that the substrate absorption rate at Z_{F} is significantly higher than the actual substrate consumption rate therein, when the rRCS is sufficient, and the actual rate depends also on the substrate and microbes. Therefore also the rGrowth in Z_{F} is lower than the substrate absorption rate and corresponding biomass yield would result otherwise. As a consequence, the difference between nutrient absorption and consumption as well as related BM growth will take place in other processing units.

The FCS rate rFCS is designed to compensate the rRCS at the selected operating point of Z_{F}, such that

rFCS=rNet+rRCS

It is very beneficial for the system that as the microbes in Z_{F} with average residence time (TResZ_{F}) in Z_{F}, likely experience their fastest growth rate at Z_{F}, corresponding a population doubling time TD, the rRCS and rFCS are selected such that said TResZ_{F} is shorter than TD, as it encourages flocking and results in lower amount of planktonic cells.

Also, as the growth phase of the microbes is interfered, the anabolic consumption of substrate is shifted to catabolic, resulting in reduced new biomass generation, yet as the cycle time is kept sufficiently short, the microbes do not reduce their energy consumption to the level typical for endogenous phase.

The minimum rRCS required to absorb majority of influent substrate in Z_{F} depends on the influent substrate concentration. The TD will increase if the rRCS and rFCS is increased and thus TResZ_{F} is decreased to the level where the substrate becomes already scarce in Z_{F}, and thus depending on other system parameters, the rRCS and rFCS can be used as a tool to adjust the TD and substrate concentration in Z_{F}.

If the total BM in Z_{F} is mZ_{F}, then typically to improve settling properties of biomass and also to reduce the amount of generated new biomass, as TResZ_{F} should be lower than TD, the approximate guidelines should be used:

rFCS>mZ_{F}/TD, and

rRCS>mZ_{F}/TD−rNet

When the rRCS and rFCS are increased or either influent flow rate or substrate concentration is decreased, the TD tends to increase and the substrate concentration in Z_{F} tends to decrease, both in a non-linear fashion.

The influent substrate concentration, the BM yield of targeted microbes and selected food to microorganism ratio (F/M) as well as a measured substrate absorption rate rSA at Z_{F} can be used to evaluate the target values for rRCS and rFCS, when such parameters are known for the system conditions applied.

However, for example in a phased degradation of an influent substrate, not all the biomass is able to degrade directly the said substrate, and thus the F/M ratio in the Z_{F} for the portion of microbes in its BM capable of degradation of said substrate is less than unity.

Thus the F/M ratio selected for the whole system differs from the F/M ratio observed in Z_{F} for any substrate present at the influent and strongly depends on the influent substrates, the spectra of microbial populations present and the system configuration. Thus, using the F/M ratios or rSA is most feasible when the system is processing a known influent with known microbes, such as production of desired end product or production of microbial biomass.

Generally, increasing the FCS and RCS rate increases the system performance until at least a local maximum is reached. Increasing the rFCS and rRCS beyond such maximum imposes system design challenges greater than the advantages achieved.

Study Results

The study was performed using different configurations of the system of this invention using activated sludge as biomass, and a normal AS process reactor was used as a reference.

The Reference Reactor (C)

The reference reactor performance was observed to be normal and similar to those currently used widely for waste water treatment. The HRT of the reactor was varied from 8 to 43 hours.

The tested reactors of this invention (types A and B)

Two kind of configurations were studied in this study, while several modifications to the configurations were tested. The configuration (A) was a 3 unit reactor with sludge circulation, and configuration (B) was a 4 unit reactor with sludge circulation.

Both (A) and (B) were tested mainly with 6 and 8 hour HRT, respectively, where the aerated portion of the reactors represented about ⅔ of the total volume and HRT. The remaining ⅓ was used to separate sludge in a settler portion of a vessel and thus sufficiently reducing the flow of the sludge to the next unit in the plug flow configuration. This separation was implemented using normal settler. Also the processing clarifier configuration of this invention was tested instead of normal settler, where the aeration of said processing clarifier was implemented mainly for mixing purposes, and thus the amount of aeration was significantly lower than in the main reactor part.

Sludge Circulation

The FCS was implemented using a peristaltic pump, which moved sludge from the first unit to the last unit in the configuration.

The sludge movement can occur either without any thickening of sludge, in which case the process fluid with the sludge was moved through the pump. Alternatively, a settling or other thickening method can be used prior to the pumping, to reduce the amount of process fluid passing directly from the first unit to the last unit for same amount of sludge moved.

During the testing, the configurations were tested both with and without sludge thickening, and the method used for sludge thickening was settling.

The settling method was tested in 2 ways. Firstly, by stopping the aeration of the first unit for 4-8 minutes and pumping the sludge settled in the bottom of the first unit, and secondly, adding a small unaerated collecting unit inside the first unit. The collecting unit was open in the top thus enabling the sludge in the first unit to enter the collecting unit, and then settling to the bottom of said collecting unit, from where it was pumped.

Also, a configuration with 3 units with sludge circulation (type A) followed with 1 similar unit without sludge circulation was tested (type Bmod). Furthermore, a 3 unit configuration (similar to type A) was tested without sludge circulation. In this configuration both FCS and RCS was used only in the first three vessels, and the fourth vessel was not participating to the BM circulation.

Furthermore, a configuration (type Z) was tested, where 6 processing units similar to those of FIG. 9 formed the reactor, such that the 4 first processing units formed the processing part ZF, Z2, Z3, ZL, and the 2 following processing units formed the processing clarifier part Za1, Za2. In the said type Z configuration, a FCS was used without any sludge thickening method by pumping ZF liquid to ZL using a peristaltic pump, whereas the processing units similar to FIGS. 8 and 9 have an intrinsic property of pumping the sludge against the process fluid flow from any processing unit to the previous processing unit.

The sludge circulation rate (SCR) can be defined for the sludge in the configuration participating to the sludge circulation, as the average time of sludge passing through all the units or processing units of said circulation and returning to the same unit or processing unit of its origin.

The SCRs used in the study varied from 0.5 to 5 days.

Tested Influent

The influent used in the test was synthetic waste water, which was designed to simulate normal municipal waste water. The COD of the influent was varied from 300 to over 4000 mg/l. This corresponded a F/M ratio range for (A) and (B) of about 0.2 to 5, and for (C) about 0.1 to 0.65. Also the other compounds, such as nitrogen and phosphorous, were varied such that the C:N:P ratio varied from 100:5:0.5 to 100:20:5.

The influent COD concentration was varied in such way, that the system had time to settle for the new influent for 10-90 hours. The HRT of (C) was mainly significantly higher than for (A) and (B), especially when exceeding the normal measurement interval, thus the (C) had also longer adaptation period.

The influent COD values beyond about 4000 were not tested, due to aeration limitations of the test vessels used. The (A) and (B) did not show any limitations of performance at high COD other than when limited by aeration.

Results

The reference reactor performed as expected, and its performance was similar to those used in municipal and other waste water treatment plants. As expected, at higher influent COD (>1500 mg/l) and high variations of influent COD, the (C) reactor could no longer reach acceptable effluent performance without significant increase of HRT.

The tested (A) and (B) produced stable good quality effluent regardless of strong variations and/or levels of influent COD.

The COD removal rate of C was mainly around 80-90%, depending on the influent. The removal rate of (C) was originally targeted to be kept in the same range as those of (A) and (B) by increasing the HRT of (C), but it turned out to not be feasible, especially at high influent COD (>1500), and thus the effluent COD of (C) was allowed to be significantly higher than those of (A) and (B) at high influent COD.

Rating the performance of various types and configurations tested, the following formula was used:

$${Effluent}_{COD} ( \frac{mg}{l} ) = X * \frac{{Influent}_{COD ( \frac{mg}{l} )}}{( HRT ( h ) * MLSS ( \frac{g}{l} ) )}$$

The X in the equation can be used as a figure of merit, such that a lower value of X indicates better removal rate, and thus also better performance of the system.

The value X for the (C) remained mainly around 8 during the tests. The X for the (A) and (B) was mainly below 2 in all variations. With optimized FCS and RCS the X was observed to be below 1. These results were obtained from influent COD values of 500 mg/l or higher.

It was also observed, that a configuration (type Bmod) where a system of this invention (type A) followed by another aerated unit not participating to the sludge circulation was tested, the processing unit not participating to the sludge circulation was not able to improve the total effluent quality, but as it represented ¼ of the total HRT, the X-values for such configuration were about ⅓ higher than for a normal type A configuration.

Also it was observed that a configuration resembling type A configuration but where the FCS and RCS were disabled completely, thus not having sludge circulation of this invention, was inferior in performance and also was unable to produce effluent with sufficient settling properties. Thus, the invention also improves also the settling properties of the MLSS at high F/M ratios.

A processing clarifier integrated in the (B) configuration was also tested as system of type Z. The total HRT of 6 hours represented the combined HRT of the whole system. The X values obtained were below 1, typically between 0.2 and 0.8, simultaneously reducing effluent TSS to about 30-200 mg/l. Depending on the partitioning between aerated portion of a processing unit and unaerated portion of a processing unit forming the channel between two aerated processing units, the TSS reduction of 90-99% was achieved in 2 processing units.

Thus the study shows, that the system of the invention can improve traditional AS system performance by a factor of 4 or higher with influent COD values >500, and an improvement factor above 10 can be reached. Also the dynamic performance during shock loading such as 2:1 loading change was excellent. This performance improvement can be translated into shorter HRT or improved effluent quality, or a combination of these.

1. Bioreactor for treating a water fluid by microbes, for producing a desired end product by biomass and/or for producing biomass, wherein the bioreactor comprises:
at least a first processing unit, a second processing unit, and a last processing unit, in a plug flow configuration,
at least one forward circulation system for circulating biomass from the first processing unit, to the last processing unit, and
at least one reverse circulation system for circulating biomass from the last processing unit to the second processing unit and from the second processing unit to the first processing unit in the plug flow configuration,
wherein a biomass flow rate of the at least one reverse circulating system is arranged to exceed a leakage flow rate of biomass along with the normal process water fluid flow between the processing units.

2. The bioreactor according to claim 1, wherein the bioreactor comprises at least four processing units.

3. The bioreactor according to claim 1, wherein the bioreactor comprises at least one biomass modification unit.

4. The bioreactor according to claim 1, wherein the bioreactor further comprises at least one additional processing step between the first processing unit and the last processing unit wherein the at least one additional processing step at least partly participates in processing of the water fluid but does not participate in a main biomass circulation.

5. The bioreactor according to claim 1, wherein the at least one processing unit comprises at least one internal clarifying unit.

6. The bioreactor according to claim 5, wherein the at least one internal clarifying unit comprises one or more controlling channels for self-adjusting water level, and water fluid flow, and reverse circulating system of biomass.

7. The bioreactor according to claim 1, wherein the bioreactor comprises at least two forward circulation systems.

8. The bioreactor according to claim 1, wherein bioreactor (BR) further comprises at least one processing clarifier unit after the last processing unit.

9. The bioreactor according to claim 1, wherein at least two processing units have been arranged at least partly in a same vessel.

10. The bioreactor according to claim 1, wherein the water fluid comprises gas.

11. A method for manufacturing a bioreactor for treating water fluid by microbes, for producing a desired end product by biomass and/or for producing biomass, wherein the bioreactor is arranged:
at least a first processing unit, a second processing unit, and a last processing unit, in a plug flow configuration,
at least one forward circulation system for circulating biomass from first processing unit to the last processing unit, and
at least one reverse circulation system for circulating biomass from the last processing unit to the second processing unit and from the second processing unit to the first processing unit (Z1) in the plug flow configuration,
wherein the biomass flow rate of the at least one reverse circulating system is arranged to exceed a leakage flow rate of biomass along with the normal process water fluid flow between the processing units.

12-16. (canceled)

17. The bioreactor according to claim 1, wherein the bioreactor further comprises at least one additional processing step between the first processing unit and the last processing unit wherein the at least one additional processing step at least partly participates in processing of the water fluid but does not participate in the forward circulation system.

18. The bioreactor according to claim 1, wherein the bioreactor further comprises at least one additional processing step between the first processing unit and the last processing unit wherein the at least one additional processing step at least partly participates in processing of the water fluid but does not participate in the reverse circulation system.

19. The bioreactor according to claim 1, wherein the bioreactor comprises at least two reverse circulation systems.

20. A method for treating water fluid by microbes, the method comprising:
treating the water fluid by microbes in a plug flow configuration in the at least a first processing unit, a second processing unit, and a last processing unit,
circulating biomass in at least one forward circulation system from the first processing unit to the last processing unit, and
circulating biomass in the plug flow configuration in at least one reverse circulation system from the last processing unit to the second processing unit and from the second processing unit to the first processing unit,
wherein a biomass flow rate of the at least one reverse circulating system is arranged to exceed a leakage flow rate of biomass along with the normal process water fluid flow between the processing units.

21. The method according to claim 20, wherein the water fluid comprises at least one of the fresh waters, process waters, waste waters, biomass, and gas.

22. The method according to claim 20, wherein the bioreactor is used for producing one of a desired end product by biomass and producing biomass.

23. The method according to claim 20, wherein the bioreactor is used for producing desired end product, wherein the desired end product comprises at least one of methane, ethanol, and microbial biomass.

24. The method according to claim 20, wherein the bioreactor is used for performing microbially a chemical reaction between at least two chemical compounds provided to the system.

25. The method according to claim 20, wherein the bioreactor is used for a bioreactor treating biomass present at the water fluid.

26. The method according to claim 20, wherein circulating biomass in at least one forward circulation system comprises circulating biomass from the first processing unit at an average rate exceeding a rate of growth of biomass in the first processing unit subtracted by a rate of disposal of biomass from the first processing unit.

27. The method according to claim 20, wherein the biomass flow rate of the at least one reverse circulating system between any two of the processing units included in the biomass circulation loop exceeds the leakage flow rate of biomass along with the normal process water fluid flow rate between the any two processing units.
